(19) United States
(12) Patent Application Publication
(10) Pub. No.: US 2009/0264865 A1
(43) Pub. Date: Oct. 22, 2009

(54) INSERTION ASSISTING TOOL FOR CATHETER, CATHETER ASSEMBLY, AND CATHETER SET

(75) Inventor: Jun KAWAI, Otsu-shi (JP)

Correspondence Address: ARENT FOX LLP, 1050 CONNECTICUT AVENUE, N.W., SUITE 400, WASHINGTON, DC, 20036, US

(73) Assignee: GOODMAN CO., LTD., Nagoya-shi (JP)

(21) Appl. No.: 12/491,847

(22) Filed: Jun. 25, 2009

(30) Foreign Application Priority Data
Dec. 11, 2006 (JP) 2006-333762

(51) Int. Cl. A61M 25/09

(52) U.S. Cl. 604/528

(57) ABSTRACT

An insertion assisting tool inserted into a catheter and positioned ahead of the catheter is provided so as to assist an insertion of the catheter. The insertion assisting tool includes: a distal end portion whose end is tapered and which has a guide wire lumen opening at a tip end and a proximal end of the distal end portion; and a shaft extending from a part of a proximal-end surface of the distal end portion to a proximal end of the insertion assisting tool.

### BACKGROUND OF THE INVENTION

This application claims the benefit of Japanese Patent Application Number 2006-333762 filed on Dec. 11, 2006, the entirety of which is incorporated by reference.

1. Field of the Invention

The present invention relates to an insertion assisting tool which is positioned ahead of a catheter for assisting an insertion of the catheter. The present invention also relates to a catheter assembly and a catheter set which utilize this insertion assisting tool.

2. Description of Related Art

Japanese Laid-open Patent Publication No. 08-224313 discloses a conventional insertion assisting tool for a catheter. As shown in the document, the conventional insertion assisting tool is inserted into a lumen of a catheter over the entire length of the catheter for the purpose of reinforcing the catheter. The insertion assisting tool is provided with a side hole for inserting a guide wire.

As the conventional insertion assisting tool is inserted into a catheter over the entire length of the catheter for reinforcement, the tool contacts with the whole inner surface of the catheter. For this reason, it becomes difficult to insert the insertion assisting tool into the catheter depending on combinations of materials for the insertion assisting tool and the catheter. Further, the insertion assisting tool has a side hole through which a guide wire passes from the inside to the outside of the insertion assisting tool, and the guide wire inserted into the insertion assisting tool protrudes from the side hole and extends in a direction across the axis of the catheter. The insertion assisting tool is positioned ahead of the catheter and gradually inserted into a body cavity with the guide wire being inserted into the insertion assisting tool. The guide wire is retrieved by being pulled out through the side hole of the insertion assisting tool. However, pulling out the guide wire is not easy because the guide wire protruding from the side hole gets tangled in the catheter. Furthermore, in the case where a new guide wire has to be inserted into the insertion assisting tool after the guide wire is removed through the side hole, it is necessary to insert the new guide wire into the insertion assisting tool through the side hole, which requires a difficult inserting operation. According to this configuration of the insertion assisting tool, replacing of the guide wire is difficult with the insertion assisting tool being positioned ahead of the catheter.

In view of the above, the present invention seeks to provide an insertion assisting tool which is easily inserted into and pulled out from the catheter and which is designed to allow replacement of the guide wire even if the insertion assisting tool is positioned ahead of the catheter.

The present invention has been made in an attempt to eliminate the above disadvantages, and illustrative, non-limiting embodiments of the present invention overcome the above disadvantages and other disadvantages not described above.

### SUMMARY OF THE INVENTION

According to a first aspect of the present invention, there is provided an insertion assisting tool inserted into a catheter and positioned ahead of the catheter so as to assist an insertion of the catheter. The insertion assisting tool comprises: a distal end portion whose end is tapered and which has a guide wire lumen opening at a tip end and a proximal end of the distal end portion; and a shaft extending from a part of a proximal-end surface of the distal end portion to a proximal end of the insertion assisting tool. With this configuration of the insertion assisting tool, the above primary object of the invention can be achieved.

The aforementioned insertion assisting tool may further comprises a soft tip which is softer than the distal end portion and arranged at the tip end of the distal end portion, and the soft tip may have a guide wire lumen in communication with the guide wire lumen of the distal end portion. With this configuration, it is further possible to prevent the insertion assisting tool from damaging the inner region of a vascular channel when it goes inside the vascular channel and also to lead the guide wire to a predetermined position for improving insertion ability and tracking ability of the insertion assisting tool.

According to a second aspect of the present invention, there is provided a catheter assembly comprising: the insertion assisting tool as described above; and a catheter having a distal-end opening in which a proximal-end edge of the distal end portion of the insertion assisting tool can be held. With this configuration of the catheter assembly, it is further possible to easily insert the catheter by using the insertion assisting tool without damaging the vascular channel, and the insertion and removal of the insertion assisting tool can be achieved smoothly where necessary. Further, replacement of the guide wire can be readily performed with the catheter being inserted.

In the aforementioned catheter assembly, the distal end portion of the insertion assisting tool may be harder than the distal end portion of the catheter. With this configuration of the catheter assembly, insertion ability of the catheter is improved while the degree of influence on the vascular channel is kept low.

In the aforementioned catheter assembly, a side hole may be provided in a distal end portion of the catheter. With this configuration of the catheter assembly, blood perfusion can be obtained even in combination with the insertion assisting tool.

According to a third aspect of the present invention, there is provided a catheter set comprising: the catheter assembly as described above; and a guiding catheter configured to receive and guide the catheter assembly. With this configuration of the catheter set, the guiding catheter can be placed as an outer sheath in a central vascular channel and the catheter can be placed as an inner sheath in a peripheral vascular channel. Further, while the catheter has been inserted, replacement of the guide wire can be performed with improved insertion ability of the catheter.

According to the present invention, the insertion assisting tool which includes a tapered distal end portion having a guide wire lumen, and a shaft extending from the distal end portion is provided, and the catheter assembly and the catheter set which utilize this insertion assisting tool are also provided. Therefore, the insertion assisting tool can be smoothly inserted into and retrieved from the catheter while assisting the insertion of the catheter. Further, replacement of the guide wire can be readily performed, and difference in level on the surface between the catheter assembly and the guide wire can be reduced so that the distal end of the catheter can be safely guided to a target site without any difficulty.

### BRIEF DESCRIPTION OF THE DRAWINGS

The above aspect, other advantages and further features of the present invention will become more apparent by describing in detail illustrative, non-limiting embodiments thereof with reference to the accompanying drawings, in which:

FIGS. 1A and 1B are explanatory views according to one embodiment of the present invention, in which FIG. 1A shows an insertion assisting tool, and FIG. 1B shows a catheter;

FIG. 2A is a sectional view of the insertion assisting tool;

FIG. 2B is a sectional view of the catheter when assembled with the insertion assisting tool;

FIG. 3A is an enlarged sectional view of a distal end portion of the insertion assisting tool shown in FIG. 1A;

FIG. 3B is an enlarged sectional view of a shaft extending from the distal end portion of the insertion assisting tool;

FIG. 4 is an enlarged sectional view taken along the line A-A of FIG. 3A;

FIG. 5A is an explanatory view of the catheter;

FIG. 5B is an enlarged sectional view partly showing the catheter of FIG. 5A;

FIG. 5C is an explanatory view showing a modification of the catheter;

FIG. 6 is an explanatory view showing a catheter assembly according to one embodiment of the present invention; and

FIG. 7 shows a use state of a catheter set according to one embodiment of the present invention.

### DETAILED DESCRIPTION OF THE EXEMPLARY EMBODIMENT

With reference to the accompanying drawings, one exemplary embodiment of the present invention will be described below.

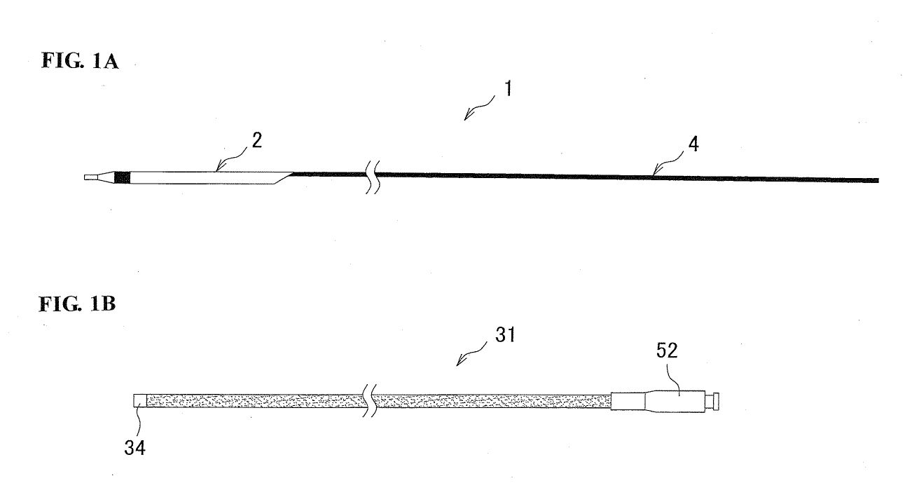

As seen in FIG. 1A, an insertion assisting tool 1 includes a distal end portion 2 positioned at a distal-side thereof, and a shaft 4 extending from the distal end portion toward a proximal-side of the insertion assisting tool 1 to occupy a great part of the insertion assisting tool 1 for its length. The insertion assisting tool 1 has a sufficient length to be inserted into a catheter 31 to be described later in a state that a part of a tapered portion 12 thereof protrudes from a distal-end opening of the catheter 31 and a proximal end of the shaft 4 protrudes from a proximal-end opening of the catheter 31. To be more specific, the length of the distal end portion 2 is approximately 200 mm, and the length of the shaft 4 (excluding the length overlapping with the distal end portion 2) is approximately 1300 mm.

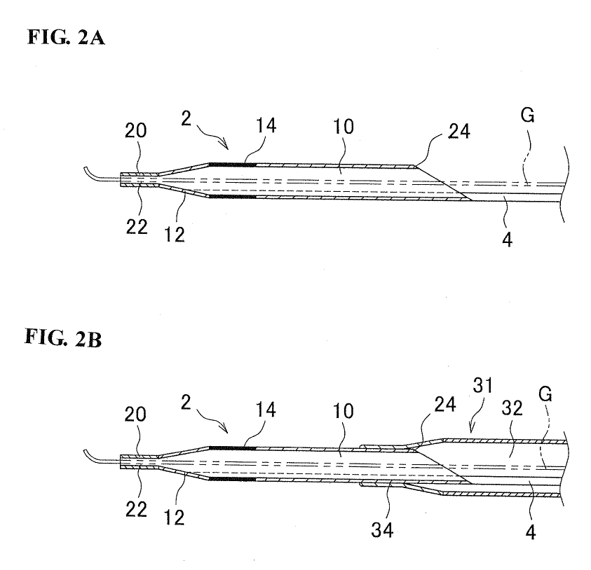

As seen in FIG. 2A, the distal end portion 2 is in a tubular shape and has a lumen 10 for a guide wire G (i.e., guide wire lumen) which opens at a tip end and a proximal end of the distal end portion 2. A tapered portion 12 is provided at the distal-end side of the distal end portion 2, and a tubular marker 14 which blocks radiation (X rays) is provided just behind the tapered portion 12. The outer surface of the marker 14 and the outer surface of the distal end portion 2 constitute a continuous surface. The outer surface of the marker 14 and the outer surface of the distal end portion 2 positioned behind the marker 14 have an outer diameter substantially the same as the diameter of the distal-end opening of the catheter 31. To be more specific, the outer diameter of the marker 14 and the distal end portion 2 is substantially the same as the inner diameter of a 5-Fr (French) catheter. Namely, the outer surface of the marker 14 and the outer surface of the distal end portion 2 positioned behind the marker 14 (at the proximal-side) have the outer diameter in the range of 1.40-1.50 mm.

A tubular-shaped soft tip 20 is made of a material which is softer than the distal end portion 2, and connected at the distal-side of the tapered portion 12. The connection is made by fusing the soft tip 20 to the distal end portion 2. The soft tip 20 also has a lumen 22 for the guide wire G. The diameter of the lumen 22 is the same as that of the lumen 10 at the distal end of the tapered portion 12. The lumen 22 is longitudinally coaxial and in communication with the lumen 10. The diameter of the lumen 22 and the distal-end opening of the tapered portion 12 is slightly larger than the diameter of the guide wire G so that the guide wire G can be slidably supported. More specifically, the diameter of the lumen 22 and the diameter of the distal-end opening of the tapered portion 12 are in the range of 0.40-0.42 mm.

A tubular marker (not shown) which blocks radiation (X rays) is provided so as to be covered by the soft tip 20.

The distal end portion 2 is made of poly(ether-block-amide) copolymer (PEBAX®) and the shore hardness thereof is 63 durometer (D). The soft tip 20 is made of PEBAX® and the shore hardness thereof is 59 D which is softer than that of the distal end portion 2. Use of PEBAX® enables the distal end portion 2 and the soft tip 20 to be easily manufactured. It should be noted that the distal end portion 2 is coated with methyl vinyl ether (VEMA) which has lubricity when being wetted, for example. In this embodiment, coating of hydrophilic polyethylene oxide (PEO) is applied to the distal end portion 2.

The opening of the lumen 10 is also positioned at the proximal-end of the distal end portion 2, being adjacent to a proximal-end surface 24 which is formed by the proximal-edge surface (i.e., O-shaped area formed by the edge surface) of the wall of the distal end portion 2. The proximal-end surface 24 is tilted with respect to the central axis of the distal end portion 2. The shaft 4 connected to a tail-end portion of the proximal-end surface 24 extends toward the proximal end of the insertion assisting tool 1.

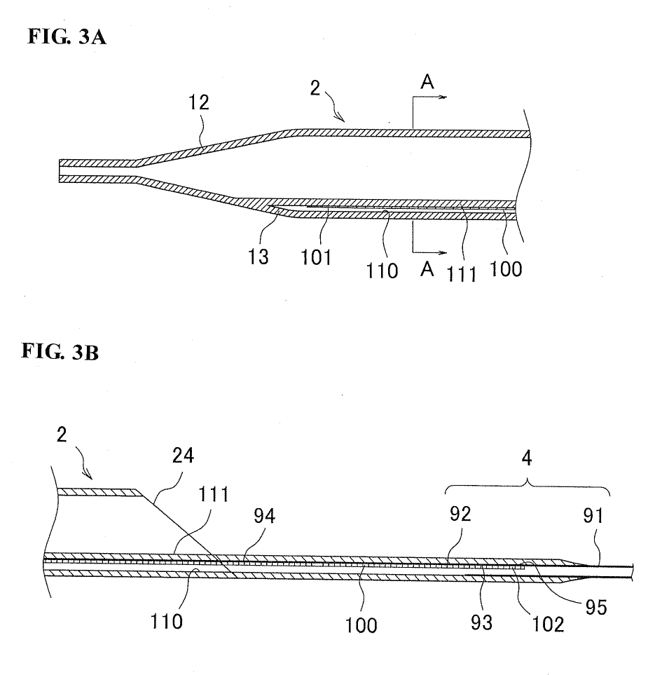

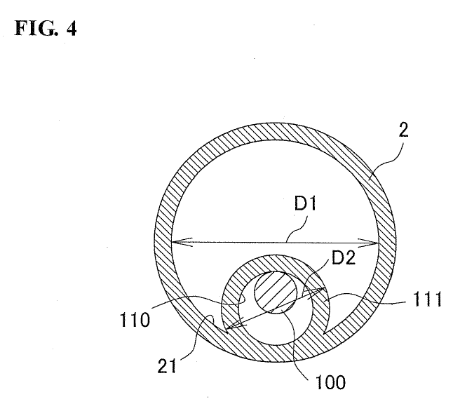

As seen in FIGS. 3A, 3B and 4, the shaft 4 is rod-shaped and connected with the side walls of the distal end portion 2. The shaft 4 is configured to be more flexible at its distal end and less flexible at its proximal end.

To be more specific, the shaft 4 is arranged at the proximal-side of the distal end portion 2, and configured to include a stainless steel hypotube 91 having an outer diameter smaller than the inner diameter D1 of the distal end portion 2, and a resin tube 92 connected to the hypotube 91 and extending toward the tapered portion 12. The resin tube 92 is made of polyamide and the outer diameter thereof is substantially the same as that of the hypotube 91. The hypotube 91 is connected to the tube 92 by being inserted and fused its distal end portion 93 into the tube 92. The hypotube distal end portion 93 is more flexible than the proximal-side of the hypotube 91, because a helical slit is provided in the hypotube distal end portion 93.

The shaft 4 is connected to the distal end portion 2 by fusing a distal edge portion 94 of the tube 92 to the wall of the distal end portion 2. Further, the insertion assisting tool 1 includes a core member 100 of which one end 101 is positioned at the distal end portion 2 and the other end 102 is positioned at the shaft 4, so that the core member 100 extends between the distal end portion 2 and the shaft 4. To be more specific, the core member 100 is inserted into the tube 92 of the shaft 4 such that the one end 101 is loosely inserted into an insertion hole forming member 111, which forms a core member insertion hole 110 extending from a proximal end 13 of the tapered portion 12 of the distal end portion 2 to a distal-end opening of the distal end portion 2. The insertion hole forming member 111 is specifically a narrow resin tube, and the core member insertion hole 110 is formed inside the distal end portion 2 by fusing the insertion hole forming member 111 to the inner wall 21 of the distal end portion 2. On the other hand, the other end 102 of the core member 100 is inserted into the hypotube 91 and fused to an inner wall 95 of the hypotube 91. The core member 100 is made of a stainless wire whose diameter becomes smaller from the other end 102 toward the one end 101.

A fluororesin coating is applied to the shaft 4. Although not shown in the drawings, a marker is provided at the proximal edge portion (i.e., a portion around the proximal edge of the welded portion) of the proximal-end surface 24.

Explanation will be given on the catheter 31 according to one exemplary embodiment of the present invention, which is used in combination with the insertion assisting tool 1. As seen in FIGS. 1B, 2B, and 5A, the catheter 31 is in a tubular shape and sized to have 5 Fr (French) inner diameter, and has a lumen 32 which opens at a tip end and a proximal end of the catheter 31. The guide wire G or other medical devices such as a balloon catheter can be inserted into the lumen 32.

The catheter 31 is capable of passing through the interior of a stent which has the inner diameter in the range of 2.25-3.50 mm after expansion. The catheter 31 is also capable of passing through a lumen of a 6-Fr guiding catheter.

A distal edge portion of the catheter 31 is provided with a soft tip 34, which is made of a material softer than that of the proximal-side of the catheter 31. The soft tip 34 is made of PEBAX® of which the shore hardness is 33 durometer (D). Thus, the soft tip 34 is softer than the distal end portion 2 of the insertion assisting tool 1. A distal edge portion of the soft tip 34 has a diameter slightly smaller than that of a proximal portion of the soft tip 34 to provide a slightly-tapered portion. The distal-end opening of the catheter 31, that is, the slightly-tapered portion of the soft tip 34 contacts with the outer periphery of the proximal-end edge of the distal end portion 2 of the insertion assisting tool 1 so as to surround and support the proximal-end edge of the distal end portion 2. The soft tip 34 also contains a radiopaque material, such as tungsten or bismuth oxide, so as to function as a radiopaque marker.

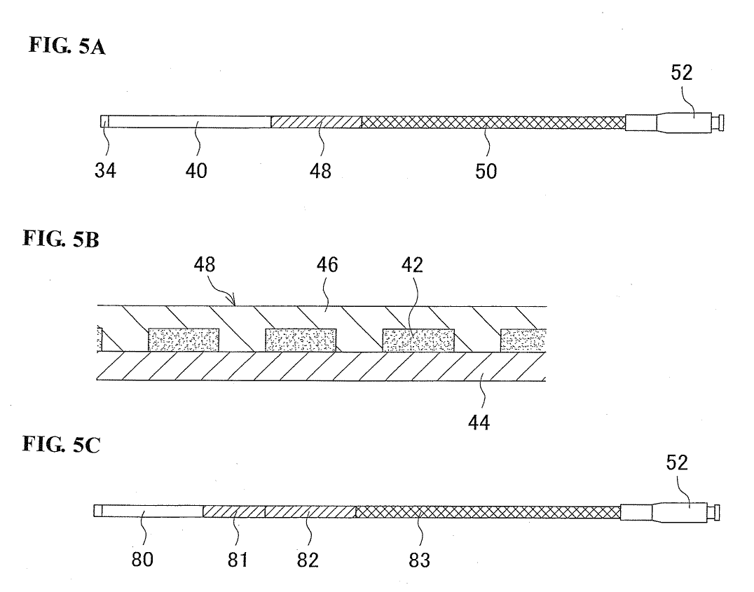

Except for the soft tip 34, the catheter 31 is configured such that the shore hardness thereof increases in a stepwise manner from the distal-side toward the proximal-side while keeping flexibility. To be more specific, the catheter 31 includes three tubular structural members such that the shore hardness increases in a stepwise manner from the distal-side. As best seen in FIG. 5A, a PEBAX® tube 40, where the shore hardness thereof is 40 D, is disposed on the proximal-side from and adjacent to the soft tip 34. Further, a tubular structural member 48 is disposed on the proximal-side from and adjacent to the tube 40. As seen in FIG. 5B, the tubular structural member 48 consists of an inner tube 44 which is made of fluororesin (polytetrafluoroethylene; PTFE), a PEBAX® outer tube 46, and a stainless helical member 42 held between the inner tube 44 and the outer tube 46. Furthermore, a tubular structural member 50 is disposed on the proximal-side from and adjacent to the tubular structural member 48. The tubular structural member 50 is a mesh-reinforced nylon tube and the shore harness thereof is 74 D. The tube 40 and the tubular structural member 48 are fused together, and the tubular structural members 48 and 50 are fused together as well. A connecting portion 52 is provided at the proximal-side of the tubular structural member 50, and various equipments are connected to the catheter 31 through the connecting portion 52. The length of the catheter 31 excluding the connecting portion 52 is approximately 1260 mm.

Fluororesin finishing (e.g., polytetrafluoroethylene coating) is applied to the inner surface of the catheter 31, while PEO coating is applied to the outer surface of the catheter 31.

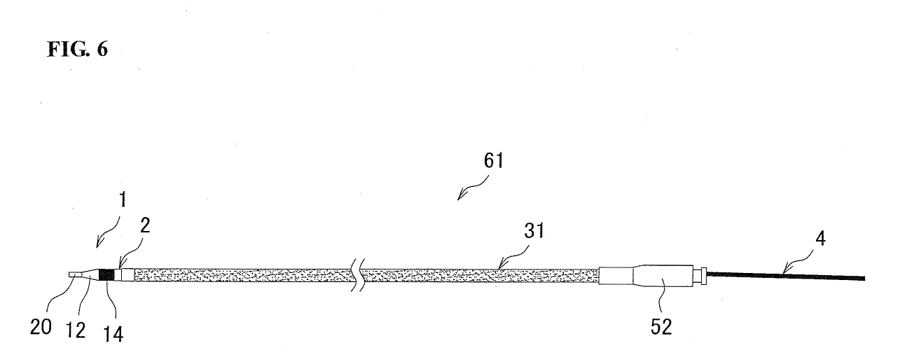

As seen in FIGS. 2B and 6, when the insertion assisting tool 1 and the catheter 31 are assembled such that the proximal-end edge of the distal end portion 2 of the insertion assisting tool 1 is inserted into the distal-end opening (distal edge portion) of the catheter 31, the tapered portion 12 of the insertion assisting tool 1 is positioned ahead of the catheter 31 and the shaft 4 penetrates through the lumen of the catheter 31 so that the proximal end of the shaft 4 protrudes from the connecting portion 52 of the catheter 31. This assembled state is a so-called normal state (insertion state) of the catheter assembly 61.

In the normal state, the catheter assembly 61 allows the guide wire G to be inserted into a remaining lumen; the remaining lumen is a remaining space to be defined by removing a space occupied by the shaft 4 from the lumen 32 of the catheter 31 extending between the proximal-end surface of the distal end portion 2 and the proximal end of the catheter 31. In order that the lumen 10 of the distal end portion 2 is capable of receiving the guide wire G inserted into the remaining lumen, the proximal-side opening of the distal end portion 2 opens toward the proximal-side of the catheter 31. In the normal state of the catheter assembly 61, the remaining lumen and the lumen 10 of the distal end portion 2 are coaxially communicated with each other.

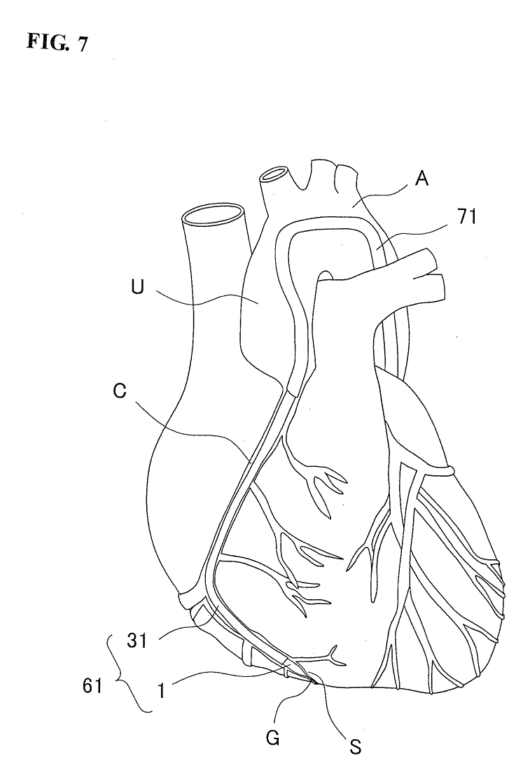

Next, description will be given on a catheter set according to one exemplary embodiment of the present invention. The catheter set comprises the catheter assembly 61 (i.e., the insertion assisting tool I plus the catheter 31), and a 6-Fr guiding catheter 71 (see FIG. 7) for receiving the catheter 31.

With reference to the drawings and in particular to FIG. 7, two usage examples of the insertion assisting tool 1, the catheter assembly 61, and the catheter set as described above will be explained. These two examples concern an approach from an aortic arc A to a peripheral stenosis lesion in a coronary artery C through an ascending aorta U.

As a first example, a rapid exchange (Rx) process of the guide wire G will be described. In order to perform the rapid exchange (Rx) process of the guide wire G, the user inserts the guiding catheter 71 of the catheter set until the distal end of the guiding catheter 71 reaches an ostium of the coronary artery C and place the guiding catheter 71 in an intended position. The user then inserts the catheter 31 into the lumen of the guiding catheter 71.

During the insertion of the catheter 31 into the guiding catheter 71, the user can smoothly insert the catheter 31 in such a way that the catheter 31 slides on the inner surface of the guiding catheter 71 because coating is applied on the outer surface of the catheter 31.

When the distal end of the catheter 31 reaches a proximity of the distal end of the guiding catheter 71, the user stops inserting the catheter 31, and then inserts the guide wire G into the lumen of the catheter 31 until the distal end of the guide wire G reaches a proximity of a lesion S in the coronary artery C.

The user then inserts the proximal-end edge of the guide wire G into the lumens 10, 22 of the insertion assisting tool 1. Thereafter, the user gradually inserts the insertion assisting tool 1 along the guide wire G so that the insertion assisting tool 1 slides along the lumen of the catheter 31. The insertion assisting tool 1 is guided along the guide wire G and inside the lumen 32 when the user manipulates the insertion assisting tool 1 using the shaft 4.

During the insertion of the guide wire G and the insertion assisting tool 1 into the catheter 31, the user can smoothly insert the guide wire G and the insertion assisting tool 1 in such a way that they slide on the inner surface of the catheter 31 because the coating is applied on the inner surface of the catheter 31.

Further, because the tapered portion 12 having an opening slightly greater than the diameter of the guide wire G is provided at the distal end portion 2 of the insertion assisting tool 1 or the soft tip 20 having the lumen 22 slightly greater than the diameter of the guide wire G is connected to the distal end portion 2 of the insertion assisting tool, the user can smoothly insert the insertion assisting tool 1 into the lumen 32 of the catheter 31 with the insertion assisting tool 1 being guided by the guide wire G and without causing the distal end portion 2 to deflect in the lumen 32 of the catheter 31. Further, the coating is applied on the outer surface of the insertion assisting tool 1 so that the user can smoothly insert the insertion assisting tool 1 in such a way that it slides in the lumen 32 of the catheter 31. The coating is also applied to the lumen 10 of the insertion assisting tool 1, so that the user can smoothly insert the insertion assisting tool 1 in such a way that it slide along the guide wire G.

The insertion assisting tool 1 includes the shaft 4 which extends from a part of the proximal-end surface 24 positioned at the proximal-end opening of the distal end portion 2. Therefore, the insertion assisting tool 1 contacts with the inner surface of the catheter 31 only at the proximal-side portion of the distal end portion 2 and at a part of the shaft 4, so that it becomes extremely easy to insert the insertion assisting tool 1 into the lumen 32 of the catheter 31. Further, the guide wire G passes through the insertion assisting tool 1 only in the lumens 10, 22 of the distal end portion 2 and the guide wire G can be inserted into and pulled out from the proximal-end opening of the distal end portion 2, so that it is possible to perform a rapid exchange of the guide wire G even with the insertion assisting tool 1.

Accordingly, the insertion assisting tool 1 and the catheter 31 are assembled into the catheter assembly 61 and set in the normal state where the tapered portion 12 of the insertion assisting tool 1 is positioned ahead of the catheter 31 and exposed from the proximal-end opening of the catheter 31. The user then inserts the catheter assembly 61 along the guide wire G until the distal end of the catheter assembly 61 reaches just before the lesion S.

According to the above embodiment, because the distal end portion 2 of the insertion assisting tool 1 is tapered, difference in level on the surface between the catheter 31 and the guide wire G is lessen so that the distal end of the catheter 31 can be safely guided to a target site without any difficulty. Further, the soft tip 20 which is softer than the distal end portion 2 is joined to the distal end of the insertion assisting tool 1. Therefore, even in the case that the catheter assembly 61 is positioned ahead of the guiding catheter 71, the catheter assembly 61 hardly injures blood vessels. Similarly, providing the soft tip 34, which is softer than the proximal-side of the catheter 31, at the distal-end opening of the catheter 31 can prevent blood vessels from being injured upon insertion of the catheter assembly 61.

According to the above embodiment, because the marker is provided at the soft tip 20 of the insertion assisting tool 1, the user can easily track a position of the tip end (distal end) of the insertion assisting tool 1. Therefore, the user can easily manipulate the insertion assisting tool 1 and the catheter assembly 61. Further, because the marker 14 is provided just behind the tapered portion 12 of the distal end portion 2, the user can easily recognize the normal state of the catheter assembly 61 by placing the distal-edge portion of the catheter 31 on the marker 14, so that it is possible to prevent the distal end of the catheter 31 from being positioned ahead of the marker 14 and protruding beyond the tapered portion 12 to cause difference in level on the surface which is apt to injure blood vessels. Furthermore, because the marker is provided at the shaft-connecting portion at the proximal-end surface 24 of the distal end portion 2, the user can easily perform replacement of the guide wire G while paying attention to a slight rise from the inner surface of the catheter 31 which corresponds to the wall thickness of the distal end portion 2 of the insertion assisting tool 1 and also to a slight rise if there is any between the distal end portion 2 and the shaft 4.

As shown in FIG. 7, when the distal end of the catheter assembly 61 reaches just before the lesion S, the user manipulates the catheter assembly 61 to push open the lesion S using the tapered portion 12 of the insertion assisting tool 1, and thereafter inserts the catheter 31 until the distal end of the catheter 31 reaches near the center of the lesion S.

During the insertion of the catheter 31, the user can gradually push open the lesion S because the distal end portion 2 of the insertion assisting tool 1 has the tapered portion 12, so that a smooth movement of the catheter 31 toward the lesion S is ensured. Even in the case that the lesion S is hardened because of calcification or the like, the user can reliably move the catheter 31 towards the lesion S. Further, the user can easily guide the catheter 31 to the lesion S even in the case that a calcified region or a stent placement region is present in front of the lesion S, because he just has to insert the tapered portion 12 into the region and if necessary he can push open the region. When the catheter 31 approaches the peripheral portion by the aid of the insertion assisting tool 1, the catheter 31 further backs up the guide wire G, so that the guide wire G can be advanced further toward the peripheral portion.

According to the above embodiment, because the catheter 31 is advanced by the aid of the insertion assisting tool 1 having the tapered distal end portion 2, the user can place the distal end of the catheter 31 deeply in the coronary artery C (Deep Engagement). Therefore, the user can firmly fix the catheter 31 or the guiding catheter 71 using a frictional force between the catheter 31 and the inner wall of the coronary artery C which occurs by deeply inserting the catheter 31 into the coronary artery C. Further, because the distal end of the catheter 31 can be advanced near the lesion S by the aid of the insertion assisting tool 1, a selective peripheral coronary arteriography is available, which requires only a small amount of contrast agent. Further, because the insertion assisting tool 1 includes the tapered distal end portion 2 and the soft tip 20 which is softer than the distal end portion 2, it is possible to prevent an intimal dissection even in the case of the deep engagement.

It is noted that the soft tip 20 is harder than the soft tip 34 of the catheter 31 for the purpose of reliably passing through the lesion S. In other words, the soft tip 34 which is not necessary to take care of passing through the lesion S, is made softer than any other parts of the catheter assembly 61, which prevents difference in level on the surface that is derived from an opening generated between the soft tip 34 and the insertion assisting tool 1 from damaging blood vessels.

Once the insertion of the distal end of the catheter 31 to the lesion S is secured, the user then manipulates the shaft 4 to pull out the insertion assisting tool 1 from the catheter 31. This pull-out operation of the insertion assisting tool 1 is as easy as the insertion operation of the insertion assisting tool 1.

The user then inserts a balloon catheter (not shown) into the catheter 31, and diagnoses or treats the lesion S. In this instance, if the user only uses the guiding catheter 71 without using the catheter 31, the guiding catheter 71 may come off from the ostium of the coronary artery C when the balloon catheter or the like is inserted into the coronary artery C. For this reason, an appropriate-sized catheter 31 is introduced into the coronary artery C as described previously so as to exert a back-up force on the balloon catheter or the like and to prevent the guiding catheter 71 from coming off. A surgery with double guiding catheters in which the outer sheath used is a 6-Fr catheter while the inner sheath used is a 5-Fr catheter, is particularly called a “five-in-six technique”. In the “five-in-six technique” and other surgery with double guiding catheters, the present invention also seeks to improve an advancing ability of the inner sheath as well as to ensure the replacement function of the guide wire G. According to the present invention, it is also possible to measure the distal-end pressure of the catheter 31 by the pull-back operation.

After diagnosis or treatment of the lesion S, the user pulls out the balloon catheter, the catheter 31, the guide wire G, and the guiding catheter 71 where necessary. According to this exemplary embodiment, inserting a single guide wire G only once is usually needed to perform operations from the insertion of the catheter 31 to the treatment of the lesion S by the aid of the insertion assisting tool 1.

As a second example, an over-the-wire process will be described. In order to perform a manipulation over the guide wire G, the user repeats the same operations as those described in the rapid exchange (Rx) of the guide wire G until the guiding catheter 71 is placed in an intended position. After the placement of the guiding catheter 71, the user inserts the catheter assembly 61 which is in the normal state into the guiding catheter 71 over the guide wire G and moves it forward to the lesion S. When the catheter 31 reaches near the center of the lesion S and the user pulls out the insertion assisting tool 1 from the catheter 31, he repeats the same operations as those described in the rapid exchange (Rx) of the guide wire G. If the guide wire G is likely to shift during insertion of the catheter assembly 61, the user needs to hold the guide wire G.

In this over-the-wire process, the following advantages can be obtained as in the case of the rapid exchange (Rx) process. Namely, it is possible to improve the advancing ability of the catheter 31 to reach the center of the lesion S, and to perform a rapid exchange of the guide wire G even with the insertion assisting tool 1 being used. Further, the insertion assisting tool 1 can be smoothly inserted into and retrieved from the catheter 31.

In the normal state, the catheter assembly 61 allows the guide wire G to be inserted into a remaining lumen; the remaining lumen is a remaining space to be defined by removing a space occupied by the shaft 4 from the lumen 32 of the catheter 31 extending between the proximal-end surface of the distal end portion 2 and the proximal end of the catheter 31. In order that the lumen 10 of the distal end portion 2 is capable of receiving the guide wire G inserted into the remaining lumen, the proximal-side opening of the distal end portion 2 opens toward the proximal-side of the catheter 31. Therefore, when the guide wire G is to be replaced while the catheter assembly 61 is in the normal state, it is possible to prevent the guide wire G from getting tangled in the catheter 31. Therefore, the user can easily perform replacement of the guide wire G.

Further, in the normal state of the catheter assembly 61, the remaining lumen and the lumen 10 of the distal end portion 2 are coaxially communicated with each other, so that when the guide wire G is inserted into the catheter assembly 61, the guide wire G can be prevented from bending in the remaining lumen and in the lumen 10 of the distal end portion 2. Therefore, the removal and insertion of the guide wire G can be achieved smoothly, and the replacement of the guide wire G can be readily performed.

The shaft 4 is configured to be more flexible at its distal end and less flexible at its proximal end. With this configuration of the shaft 4, a stress concentration resulting from the difference in flexibility between the shaft 4 and the distal end portion 2 is reduced, so that a breakage (kink) of the insertion assisting tool I can be prevented. Further, the insertion assisting tool 1 includes the core member 100 of which one end 101 is positioned at the distal end portion 2 and the other end 102 is positioned at the shaft 4, so that the core member 100 extends between the distal end portion 2 and the shaft 4. This can further reduce the possibility of generating a kink in the insertion assisting tool 1.

Other exemplified embodiments of the present invention will be described below, in which the above exemplary embodiments have been modified in various ways. According to one modification, the catheter 31 may be modified such that the shore hardness of the tubular structural member 50 (see FIG. 5A) is 70 D. As best seen in FIG. 5C, as another modification, the catheter 31 may comprise one tube 80 and three tubular structural members 81-83. As seen from the soft tip side of the catheter 31, the shore hardness of the tube 80 may be 40 D, the shore hardness of the tubular structural member 81 may be 40 D, the shore hardness of the tubular structural member 82 may be 63 D, and the shore hardness of the tubular structural member 83 may be 74 D.

A side hole may be formed in the distal end portion of the catheter 31, for example, at a region of the distal end portion except for the distal-edge portion holding the distal end portion 2 of the insertion assisting tool 1 when assembled into the catheter assembly 61 being in the normal state. To be more specific, the side hole is provided at the catheter 31 in the range of 30-150 mm away from the distal-edge of the catheter 31. Blood perfusion can be obtained by the provision of the side hole.

As an alternative, whole the catheter may be made of a single tube, a single tubular structural member with a helical member, or a mesh-reinforced single tubular structural member. Further, the insertion assisting tool, the catheter, and the guiding catheter may be modified differently from those of the above exemplary embodiments with respect to the constituent material, hardness, with or without the soft tip, with or without the marker, size, length, application purpose, etc. Further, the user may only use the catheter assembly without using the guiding catheter. In this instance, the user inserts the guide wire into the catheter while the catheter is placed in a target site. The user then introduces the insertion assisting tool into the catheter and advances the catheter assembly with the insertion assisting tool being positioned ahead of the catheter.

The shaft of the insertion assisting tool may extend from the tip end portion of the proximal-end surface of the distal end portion which is tilted with respect to the central axis of the distal end portion. Alternatively, the shaft may extend from a half-peripheral or a quarter-peripheral portion of a proximal-end surface which is normal to the central axis of the distal end portion. The shaft may be molded integrally with the distal end portion or formed by cutting out from the distal end portion. A cross-sectional shape of the shaft may be same as that of one section of the proximal-end surface. The distal-edge portion of the catheter may not be tapered.

In the rapid exchange (Rx) process mentioned above, the order of the operations may be changed where necessary; for example, the guide wire is advanced near the lesion S after the guiding catheter is placed in an intended position, the catheter is then inserted along the guide wire until the distal end of the catheter reaches the distal end of the guiding catheter, the insertion assisting tool is inserted into the lumen of the catheter along the guide wire, and after the catheter assembly in the normal state is formed, the catheter assembly is guided to the lesion S. Alternatively, each of the operations may be partly changed, omitted, or another operation may be added. For other processes such as the over-the-wire process, the order of the operations may be changed, or each operation may be partly changed, omitted, or another operation may be added.

According to the present invention, an insertion assisting tool, a catheter assembly, and a catheter set, which are the same or similar to those described in the above preferred embodiment, may be applied to other surgeries other than the “five-in-six technique”. They can be used for a biological duct other than a coronary artery including a carotid artery. Further, an insertion assisting tool which is the same or similar to that disclosed in the above preferred embodiment may be used with and positioned ahead of a delivery catheter for inserting a diagnostic device such as an ultrasound diagnostic catheter, or may be used with and positioned ahead of an aspiration catheter for aspirating blood clot, etc.

What is claimed is:

1. An insertion assisting tool inserted into a catheter and positioned ahead of the catheter so as to assist an insertion of the catheter, the insertion assisting tool comprising:
a distal end portion whose end is tapered and which has a guide wire lumen opening at a tip end and a proximal end of the distal end portion; and
a shaft extending from a part of a proximal-end surface of the distal end portion to a proximal end of the insertion assisting tool.

2. An insertion assisting tool according to claim 1, further comprising a soft tip which is softer than the distal end portion and arranged at the tip end of the distal end portion, wherein the soft tip has a guide wire lumen in communication with the guide wire lumen of the distal end portion.

3. A catheter assembly comprising:
an insertion assisting tool of claim 1; and
a catheter having a distal-end opening in which a proximal-end edge of the distal end portion of the insertion assisting tool can be held.

4. A catheter assembly according to claim 3, wherein the distal end portion of the insertion assisting tool is harder than the distal end portion of the catheter.

5. A catheter assembly according to claim 3, wherein a side hole is provided at a distal end portion of the catheter.

6. A catheter set comprising:
a catheter assembly of claim 3; and
a guiding catheter configured to receive and guide the catheter assembly.
